(19) United States
(12) Patent Application Publication
(10) Pub. No.: US 2022/0176060 A1
(43) Pub. Date: Jun. 9, 2022

(54) DISPOSABLE FILTER WITH IMPRINT

(71) Applicant: Drägerwerk AG & Co. KGaA, Lübeck (DE)

(72) Inventors: Edgar ISE, Lübeck (DE); Markus HAMPE, Lübeck (DE)

(21) Appl. No.: 17/539,928

(22) Filed: Dec. 1, 2021

(30) Foreign Application Priority Data
Dec. 4, 2020 (DE) 10 2020 132 320.4

(51) Int. Cl. A61M 16/10; B01D 46/00

(52) U.S. Cl. CPC A61M 16/106; B01D 46/009; A61M 2205/60; B01D 2273/16; A61M 2205/6081; B01D 46/0001

(57) ABSTRACT

A disposable filter (100), for use in a breathing circuit of a ventilator, includes a filter housing (110), which encloses a filter material (112) of the disposable filter. The filter housing has a first filter opening (118) and a second filter opening (119) for the respective connection of a gas-carrying (gas-guiding) component, especially of a respective tube, for a gas to be carried through the disposable filter. The filter material is arranged between the first filter opening and the second filter opening. The disposable filter has on the filter housing an imprint (120), which is arranged on the filter housing by a printing process, and which forms a structured surface (122). The structured surface can be written on and includes at least seven check (marking) areas (124).

### CROSS REFERENCE TO RELATED APPLICATIONS

This application claims the benefit of priority under 35 U.S.C. § 119 of German Application 10 2020 132 320.4, filed Dec. 4, 2020, the entire contents of which are incorporated herein by reference.

### TECHNICAL FIELD

The present invention pertains to a disposable filter for use in a breathing circuit of a ventilator. The present invention pertains, furthermore, to a process for printing on a disposable filter.

### TECHNICAL BACKGROUND

Disposable filters, which are arranged between the ventilation tube and the patient or between the ventilation tube and the ventilator in a breathing circuit, are known, in principle, from use in the area of intensive care medicine and anesthesia. These disposable filters purify the breathing gas, which goes to the patient or comes from the patient, from particles and microbes in different degrees of quality and/or assist a remoisturizing of this breathing gas. In this connection, for example, mechanical filters, electrostatic filters and/or HME (Heat Moisture Exchanger) filters are known.

Especially in case of patients ventilated over the long term, it is important to know how long such a filter has already been used at the patient and consequently to know when such a filter has to be replaced with a new filter in order to guarantee a sufficient filter action, in order to keep the pneumatic filter resistance low and in order to avoid an excessive microbial contamination of the filter.

In this case, the use of stickers, which can be glued onto a disposable filter, in order to note thereon the date and the time, at which the disposable filter was used, is known. As a result, it can always be traced back for how long the filter has been used up to a given time and whether a change of the filter is necessary based on its use time.

### SUMMARY

An object of the present invention is to provide an improved disposable filter, and in particular a disposable filter with an improved possibility of identification.

A disposable filter for use in a breathing circuit of a ventilator, with a filter housing and with an imprint on the filter housing, is provided according to the present invention for accomplishing this object.

In this case, the filter housing encloses a filter material of the disposable filter, wherein the filter housing has a first filter opening and a second filter opening for the respective connection of a gas-carrying (gas-guiding) component, especially of a respective tube, for a gas to be carried through the disposable filter. The filter material is arranged between the first filter opening and the second filter opening in this case.

The imprint on the filter housing is arranged on the filter housing by means of a printing process, especially by means of a pad printing process, and forms in the process a structured surface, wherein the structured surface can be written on, and wherein the structured surface comprises at least seven marking regions or check areas. Advantageously each marking region or check area is delimited and/or defined at least in part by indicia that is provided by the imprint. The indicia of such marking regions or check areas uniquely distinguish each of the marking regions or check areas from the other marking regions or check areas.

It was found within the framework of the present invention that the manual effort for identifying the disposable filter should be as low as possible in order to relieve health care staff from performing this regularly necessary identification. In order to solve this problem, the present invention provides an imprint on the filter housing, which can be written on. In addition, check areas make possible a comfortable identification on what day the disposable filter was used, without a date needing to be written down.

The disposable filter according to the present invention makes possible thereby a direct writing on the structured surface of the disposable filter, so that stickers do not need to be carried along by the health care staff and also do not have to be applied to the disposable filter manually.

In addition, the provision of seven check areas (imprinted marking areas) advantageously makes possible an identification of the day, on which the disposable filter was used (deployed), without a date having to be written out. As a result, the identification of the disposable filter is, moreover, especially reliable, since errors cannot occur during the writing down of the date. Thus, a person typically knows the currently present day of the week, whereas the recapitulation of the current date means a certain cognitive effort. In addition, the identification of the date can take place in a standardized manner regardless of language differences or different national special features in the way of spelling the date.

According to the present invention the seven check areas indicate seven weekdays, so that the currently present day can be displayed by marking a marking area—checking a check area—from these seven check areas (imprinted marking regions). In addition, the provision of a check (marking) area for displaying a use (deployment) day of the corresponding disposable filter makes possible an especially rapid detection of this use day, without detection of a plurality of numbers, which have to be used for the writing out of a date, being necessary. Each of the seven check (marking) areas is preferably labeled in order to ensure that all persons using the filter associate the same weekday with the corresponding check (marking) area.

A structured surface is in the sense of the present invention is structured by the surface forming a visually detectable structure. The surface is structured by the surface having the seven check (marking) areas.

The pad printing process is generally known to the person skilled in the art and will therefore not be explained in detail below. The use of the pad printing process is advantageous for the disposable filter according to the present invention to the effect that a prior-art process is utilized, which makes possible above all a reliable printing for curved surfaces. Hence, a reliable printing on and a cost-effective manufacture of the disposable filter are obtained as a result.

In alternative embodiments according to the present invention, a screen printing process or a digital printing process is used for the imprint. The ability to write on the structured surface of the imprint means that the imprint can be written on, preferably written on irreversibly. Such imprints that can be written on are known, in principle, so that prior-art characteristics of the imprint, which make possible such an ability to write on, will not be discussed below.

A ventilator in the sense of the present invention is defined as a device, which assists the ventilation of a patient to be ventilated. In addition to a conventional ventilator, this may also be, for example, an anesthesia apparatus or the like.

Preferred embodiments of the disposable filter according to the present invention will be described below.

The disposable filter according to the present invention is preferably a mechanical filter, an electrostatic filter, an HME filter or a combination of two or three of the above-mentioned filters. The use of the imprint according to the present invention for such filters, especially for an HME filter, is especially advantageous, because such disposable filters are only configured for a short use time. Thus, an HME filter shall, for example, be should already be changed after 24 hours to guarantee a sufficient filter action and to avoid an excessive contamination.

In a preferred embodiment the structured surface has such a roughness that it can be written on with a pen (the structured surface has a surface roughness configured for being written on by a pen). The ability to write on with a pen advantageously makes possible the use of any desired pen carried along. This is advantageous because hospital staff typically carry a pen with themselves. As a result, the awkward use of a writing instrument intended especially for the imprint is avoided.

In another advantageous embodiment, the structured surface comprises, in addition to the seven check (marking) areas, a round clock area, by means of which a time can be marked on the structured surface. In this embodiment, not only the date of the use of the corresponding filter can be indicated by means of one of the seven check (marking) areas, but also a time of the use of this filter can be indicated by means of a marking, a circling and/or a checking and/or otherwise marking of a part of the clock area. This is especially advantageous for a filter, which already has to be changed after a few hours. Thus, in this embodiment, the date and the time of the use of the filter can be identified especially rapidly and this information can, in addition, be optically detected especially rapidly in order to estimate whether a change of the corresponding disposable filter is necessary.

In another embodiment the structured surface is a contiguous surface. The structured surface in this embodiment can be optically detected in an especially simple manner since different areas of a non-contiguous surface do not have to be analyzed. The imprint according to this embodiment allows, moreover, an especially effective manufacture since a plurality of imprints do not have to be applied separately from one another.

In one preferred variant of the previous embodiment, the structured surface is a surface enclosing the first filter opening or the second filter opening in an at least partly circular manner. For a surface shaped in this manner, the provision of a pad printing process is especially advantageous since a manual gluing of a sticker with a circular shape would mean a certain effort for the health care staff. In addition, the provision of the pad printing process is especially advantageous since an imprint with a circular shape for a housing typically extends over an arched surface and therefore a reliable printing over such a surface in an especially simple manner is possible by means of the pad printing process. The area of the structured surface that is relevant for the identification of the use date of the filter can be detected optically especially rapidly due to the circularly extending structured surface (extending in a circular manner). Thus, a large imprint draws the attention of the user to this imprint in a more rapid manner than when, for example, an imprint has to be sought first on a lateral surface of the filter.

The structured surface especially preferably comprises a product identification for the disposable filter. In this embodiment, the identification may also be provided on the disposable filter together with the provision of the imprint for the identification of the use date. An additional production step for the application of the product identification may thus be eliminated.

The filter material is installed permanently in the filter housing due to a non-detachable structure of the filter housing in an especially preferred embodiment. According to this embodiment, the filter housing may be especially advantageously identified by means of the imprint according to the present invention. A filter with a permanently installed filter material is thus preferably a disposable product, in which no re-identification by means of the structured surface is necessary since the correspondingly identified surface is disposed of after a single use. As an alternative, the disposable filter may be a filter, in which the filter material is a disposable material, which may, however, be replaced for a future use.

In an especially preferred embodiment, the disposable filter is arranged in a separate package to be opened irreversibly before it is used for the first time. The package to be opened irreversibly is preferably a welded foil bag. As an alternative or in addition, the disposable filter has a respective closure for closing the first filter opening and/or the second filter opening before a first-time use of the disposable filter. The provision of a respective closure ensures that the filter material was first used as of the day identified on the structured surface and was not already exposed previously to a gas, for example, due to ambient air. In this embodiment, the structured surface, especially a check (marking) area, can be indicated by means of a corresponding identification, as of when the filter material was exposed to a gas for the first time.

In one advantageous embodiment, a color of the imprint indicates a filter type of the disposable filter from a predefined group of filter types. In this embodiment, the imprint also has, in addition to the identification when the filter was used for the first time, the function of making possible a rapid detection of the present filter type based on its color. Especially relevant filter types are, for example, HME filters, electrostatic filters, mechanical filters and/or a combination of a mechanical filter and an HME filter.

In an especially advantageous embodiment, the structured surface comprises an iconographic symbol, which indicates an ability to write on this surface for a user of the disposable filter. Such an iconographic symbol is, for example, a stylized pen. The provision of such an iconographic symbol makes possible a rapid optical detection of the possible ability to write on the imprint by the user of the disposable filter. Consequently, it can be rapidly detected that a surface can actually be written on, especially where a surface should be written on, so that a sticker according to the known state of the art is not used as an aid in spite the ability to write on the filter housing.

According to another aspect of the present invention, a process is provided for printing on the disposable filter, which has the following steps,
provision of a filter housing; and
printing on the filter housing by means of a printing process, especially by means of a pad printing process, such that a corresponding imprint forms a structured surface,
wherein the structured surface can be written on and wherein the structured surface comprises at least seven check (marking) areas.

The process for printing on the disposable filter is provided for accomplishing the above-mentioned object.

The disposable filter according to the present invention is manufactured by the process according to the present invention such that this process has all the advantages of the disposable filter. The process according to the present invention especially makes possible the especially simple manufacture of an imprint, by means of which a use date of a disposable filter can be noted in a rapid and simple manner, without having to glue on a sticker or having to write out a date.

Furthermore, the process makes possible the preparation of an imprint, which can be optically detected in an especially simple and rapid manner in order to decide whether the corresponding disposable filter has to be replaced.

The present invention shall now be explained in more detail on the basis of advantageous exemplary embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view of a first exemplary embodiment of a disposable filter according to the present invention;

FIG. 2 is a top view showing the disposable filter and showing an imprint of the disposable filter according to the present invention according to the first exemplary embodiment;

FIG. 3 is a top view showing a disposable filter according to a second exemplary embodiment according to the present invention and showing an imprint of the disposable filter;

FIG. 4 is a top view showing a disposable filter according to a third exemplary embodiment according to the present invention and showing an imprint of the disposable filter; and

FIG. 5 is a flow chart of an exemplary embodiment of a process according to the present invention.

### DESCRIPTION OF PREFERRED EMBODIMENTS

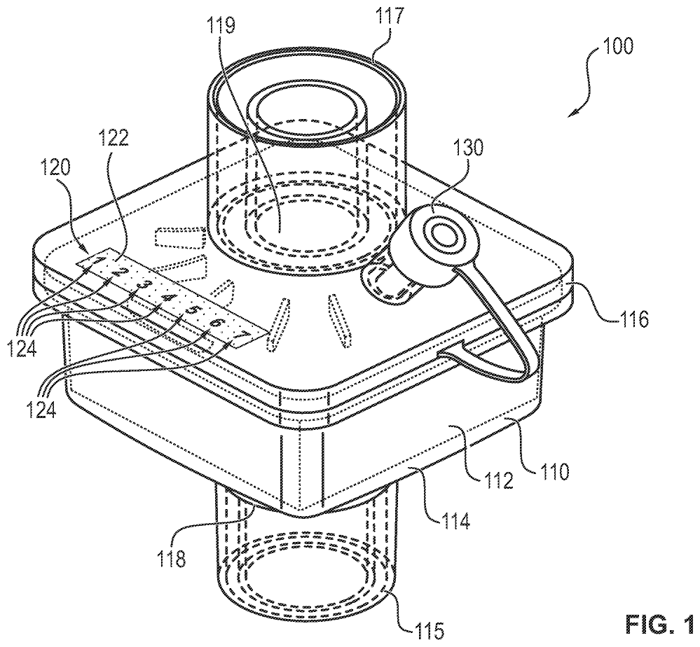

Referring to the drawings, FIG. 1 shows a perspective view of a first exemplary embodiment of a disposable filter 100 according to the present invention.

The disposable filter 100 is configured for use in a breathing circuit of a ventilator. The disposable filter 100 has, for this purpose, a common filter housing 110, which encloses a filter material 112 of the disposable filter 100. In the exemplary embodiment shown, the filter housing 110 is comprised of a first housing component 114 with a first port 115 for a tube or a Y-piece and of a second housing component 116 with a second port 117 for a gas-carrying (gas guiding) component, in the present case a tube or a device-side port. The two housing components 114, 116 are connected to one another in a positive-locking manner by means of welding, especially by means of ultrasound welding. Housing components of the filter housing in an alternative exemplary embodiment are connected to one another permanently, and especially in a non-detachable manner, by means of a bond, by means of a sealed latching mechanism or the like. A respective filter opening 118, 119 is provided in the corresponding housing components 114, 116 for providing the two ports 115, 117. In this case, the two filter openings 118, 119 are arranged such that the filter material 112 is located between the first filter opening 118 and the second filter opening 119. The two ports 115, 117 are configured in the exemplary embodiment shown with a plurality of coaxial interfaces for tubes, a Y-piece or a device-side port. The filter housing 110 is preferably made of polypropylene.

According to the present invention, an imprint 120 is provided on the filter housing 110, which is arranged on the filter housing 110 by means of a pad printing process. This imprint 120 forms a structured surface 122, i.e., a surface, which is visually subdivided into a certain structure, namely into seven check (marking) areas 124, among others, in the present case. The structured surface 122 can in this case is configured to be written on. The provision of a pad printing with a corresponding printing characteristic in order to make possible a surface that can be written on is already known and will therefore not be explained in detail below.

In the exemplary embodiment shown, the seven check (marking) areas 124 are formed by seven numbers from 1 to 7, which represent the seven days of the week. In an alternative or additional exemplary embodiment, the check (marking) areas are formed or complemented by at least one respective letter, which indicates the day corresponding to this check area or marking area.

It is possible to note rapidly and intuitively when the corresponding disposable filter 100 was used by means of the check (marking) areas shown.

The structured surface 122 can in this case be written on with a pen. The structured surface 122 has thus such a roughness whereby an ability to write on by means of a pen is possible. In particular, the structured surface has a surface roughness configured for being written on by a pen.

Furthermore, it is shown that a drainage closure 130 for closing a corresponding drainage opening is provided. This drainage closure 130 is preferably closed before a first-time use of the disposable filter 100. The additional opening offers the user via a corresponding port the possibility to suction condensation water out of the filter in order to increase a use time of the filter, since this filter does not need to be replaced after the collection of a critical quantity of water. A closure for closing the corresponding filter opening in one exemplary embodiment, not shown, is provided at least for the first port. This closure is preferably closed before a first-time use of the disposable filter.

In one exemplary embodiment, not shown, the closure is secured on the port by means of a glued seal before the first-time use. The seal ensures in this case that the closure has not inadvertently come off after manufacture, so that the disposable filter would already possibly be limited in its ability to function.

The color of the structured surface 122 preferably indicates a filter type of the disposable filter 100 from a predefined group of filter types. In the exemplary embodiment being shown, the disposable filter 100 is a mechanical filter. As an alternative or in addition, the filter may be an electrostatic filter or an HME filter. Such filter types preferably have an imprint 120 of a different color than the mechanical filter shown.

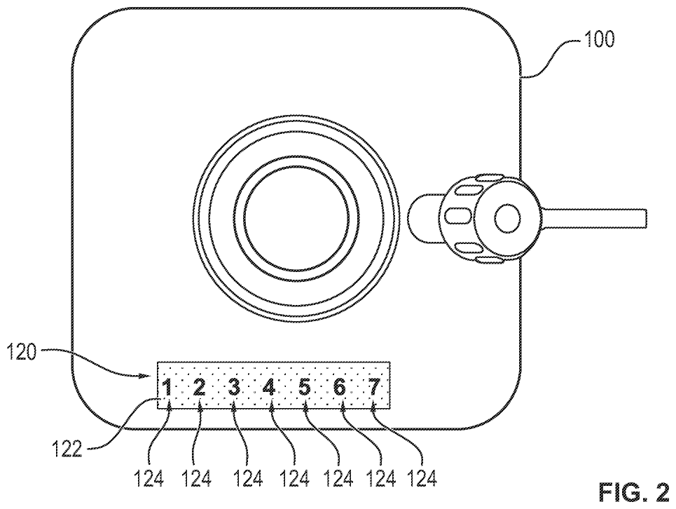

FIG. 2 shows a view of an imprint 120 of the disposable filter 100 according to the present invention according to the first exemplary embodiment.

This view illustrates that the check (marking) areas 124 can be recognized and checked (marked) in a rapid and simple manner after the disposable filter 100 was used at a patient. Furthermore, this view illustrates that a check (marking) area, which was checked, ticked, circled or marked in a similar manner, can be detected clearly by the corresponding health care staff in order to decide whether the disposable filter has to be replaced or not.

In the exemplary embodiment shown, the number 1 may correspond, for example, to Monday, and the number 7 may correspond to Sunday. Thus, it can be rapidly detected via the marked number, on which day the disposable filter 100 was used.

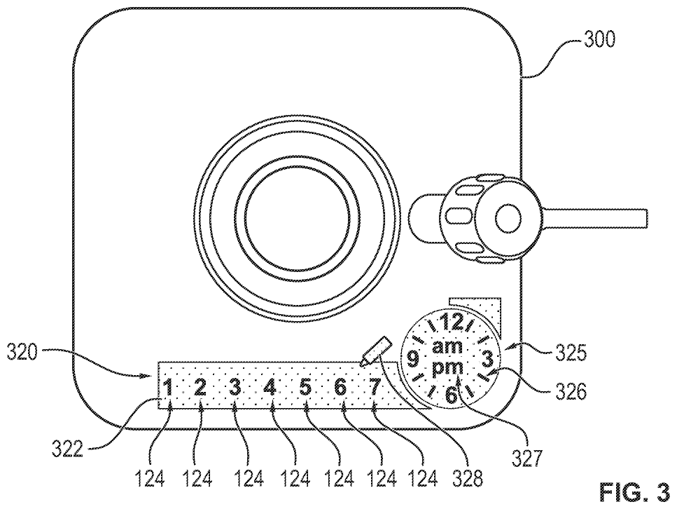

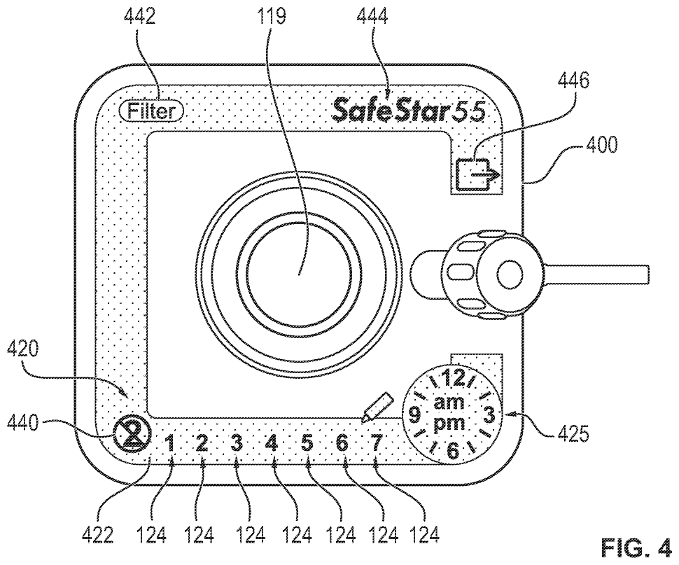

FIGS. 3 and 4 show a respective view of an imprint 320, 420 of a disposable filter 300, 400 according to the present invention according to a second exemplary embodiment and according to a third exemplary embodiment.

The disposable filter 300 shown in FIG. 3 differs from the disposable filter 100 exclusively by its imprint 320.

The imprint 320 comprises, in addition to the seven check (marking) areas 124, a round clock area 325, by means of which a time can be noted, at which the disposable filter 300 was used. The clock area 325 has, for this purpose, an hour indication 326 from 1 o'clock to 12 o'clock and a symbol, which indicates by means of the abbreviation 327 “am” and “pm” whether the time is in the morning or in the afternoon. Thus, for example, the symbol “am” may be checked or otherwise marked and, in addition, a dash may be drawn at 3:00, so that it is clear that the time is 3 o'clock in the afternoon, i.e., it is 3 pm. In addition, it is made clear by means of the marking of one of the seven check (marking) areas 124 on which day the disposable filter was used at this corresponding time.

The disposable filter 300 with its imprint 320 is therefore especially advantageous for the use of a disposable filter, which shall be used for only a few hours, especially for less than 48 hours, and preferably for less than 24 hours. For such filters, an indication, which allows a time resolution of individual hours, and not only of days, is provided as a possibility of identification on the imprint 320.

The clock area 325 is a round clock area in the exemplary embodiment shown. In an alternative exemplary embodiment, the clock area is a rectangular clock area and/or a free text field, by means of which the current time can be written down on the imprint 320. In this alternative exemplary embodiment, not shown, a greater manual effort is necessary though due to the entry of the time; however, the disposable filter in this case nevertheless has the advantage that the current data does not have to be entered by hand. This leads to a lower manual effort and additionally to a lower risk of errors when the use date is entered.

In addition, the imprint 320 has on its structured surface 322 an iconographic symbol 328, namely a stylized pen located adjacent to the check (marking) areas 124. It is indicated by this iconographic symbol 128 that the surface connected to it, i.e., the structured surface 322, can be written on for the user of the disposable filter 300.

The disposable filter 400 shown in FIG. 4 differs from the disposable filter 300 exclusively by its imprint 420.

Thus, the structured surface 422 has, in addition to the check (marking) areas 124 and to the clock area 425, a plurality of product identifications 440, 442, 444, 446. These product identifications are provided, in part, because of regional regulatory requirements. Thus, a product identification may indicate the manner of disposal, relevant ingredients of the material, an intended one-time use of the product, a product name, a manufacturer name or the like. The product identification 442 indicates, for example, that the disposable filter shown is a mechanical filter, in contrast to the HME filter in the present case. In the exemplary embodiment being shown, the disposable filter 400 shown has, for example, the product name “SafeStar 55.” The digits “55” indicate in this case a dead space volume of the filter, which amounts to 55 mL.

The structured surface 422 is in this case shaped such that the second filter opening 119 shown is enclosed by the structured surface 422 in an at least partly circular manner. The structured surface 420 is a contiguous surface in this case. Unlike the surface 320, the clock area 425 is configured as contiguous with the other areas of the structured surface 420 and is configured as only partly round.

Due to the structure of the imprint 420 enclosing in a circular manner, namely at least partially encircling, this imprint can be optically detected rapidly, so that correspondingly marked areas and/or areas to be marked of the imprint 420 are also rapidly detected by a user of the disposable filter 400.

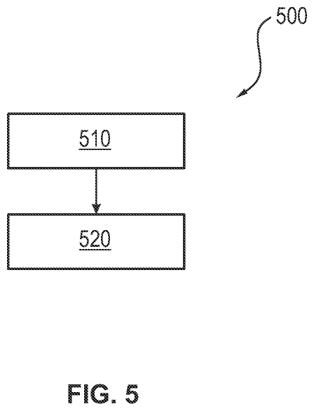

FIG. 5 shows a flow chart of an exemplary embodiment of a process 500 according to the present invention.

The process 500 according to the present invention is configured for printing on a disposable filter. The process 500 has for this purpose the steps described below.

A first step 510 comprises a provision of a filter housing.

A next step 520 comprises a printing on of the filter housing by means of a printing process, especially by means of a pad printing process, such that a corresponding imprint forms a structured surface, wherein the structured surface can be written on, and wherein the structured surface comprises at least seven check (marking) areas.

The two steps of the process 500 are carried out in the order described. Thus, first the filter housing is manufactured and then the filter housing is printed on according to the present invention by means of the pad printing process. The time interval between the two steps depends on the manufacturing process selected. At the latest before the pad printing process is carried out, the filter housing is provided for the corresponding device in the sense of step 510 in order to then make it possible to print on the structured surface by means of the corresponding device.

The process 500 according to the present invention for printing on the disposable filter may be a partial process of a manufacturing process for the corresponding disposable filter. Within the framework of this manufacturing process, typically additional steps are carried out, for example, the insertion of the filter material into the disposable filter and the like. The printing on according to step 520 is especially advantageously carried out as the last process step. As a result, the housing components for all filter variants can be configured as identical parts, before the use is specified by the insertion of the corresponding filter material, so that the printing on according to the present invention is then carried out. Such process steps may especially advantageously be carried out in an automated manner.

While specific embodiments of the invention have been shown and described in detail to illustrate the application of the principles of the invention, it will be understood that the invention may be embodied otherwise without departing from such principles.

### LIST OF REFERENCE NUMBERS

100, 300, 400 Disposable filter
110 Filter housing
112 Filter material
114 First housing component
115 First port
116 Second housing component
117 Second port
118 First filter opening
119 Second filter opening
120, 320, 420 Imprint
122, 322, 422 Structured surface
124 Check (marking) area
130 Drainage closure
325, 425 Clock area
326 Hour indication
327 Abbreviation
328 Iconographic symbol
440, 442, 444, 446 Product identification
500 Process
510, 520 Process steps

What is claimed is:

1. A disposable filter for use in a breathing circuit of a ventilator, the disposable filter comprising:
a filter housing;
a filter material, wherein the filter housing encloses the filter material of the disposable filter, the filter housing has a first filter opening and a second filter opening for a respective connection of a gas-carrying component for a gas to be passed through the disposable filter and the filter material arranged between the first filter opening and the second filter opening; and
an imprint on the filter housing, wherein the imprint is arranged on the filter housing by a printing process, and which imprint forms a structured surface, wherein the structured surface can be written on, and wherein the structured surface comprises at least seven marking regions.

2. A disposable filter in accordance with claim 1, wherein the structured surface has a roughness configured so as to be written on with a pen.

3. A disposable filter in accordance with claim 1, wherein the structured surface further comprises, a clock area by means of which a time can be marked on the structured surface.

4. A disposable filter in accordance with claim 1, wherein the structured surface is a contiguous surface.

5. A disposable filter in accordance with claim 4, wherein the structured surface is a surface enclosing the first filter opening or the second filter opening in an at least partly circularly.

6. A disposable filter in accordance with claim 1, wherein the structured surface comprises a product identification for the disposable filter.

7. A disposable filter in accordance with claim 1, wherein a color of the imprint indicates a filter type of the disposable filter from a predefined group of filter types.

8. A disposable filter in accordance with claim 1, wherein the structured surface comprises an iconographic symbol, which indicates an ability to write on the structured surface for a user of the disposable filter.

9. A disposable filter in accordance with claim 1, wherein the disposable filter is selected from the group comprising an HME filter, an electrostatic filter, a mechanical filter and combinations thereof.

10. A disposable filter in accordance with claim 1, wherein the printing process that arranges the imprint on the filter housing is a pad printing process.

11. A process for printing on a disposable filter, the process comprising the steps of:
providing a filter housing:
printing on the filter housing by means of a printing process to arrange a corresponding imprint on the filter housing, which imprint forms a structured surface, wherein the structured surface is configured to be written on, and wherein the structured surface comprises at least seven marking regions.

12. A process according to claim 11, further comprising providing a filter material, wherein:
the filter housing encloses the filter material of the disposable filter;
the filter housing has a first filter opening and a second filter opening for a respective connection of a gas-carrying component for a gas to be passed through the disposable filter;
the filter material is arranged between the first filter opening and the second filter opening; and
the printing process that arranges the imprint on the filter housing is a pad printing process.

13. A process according to claim 12, wherein the structured surface has a surface roughness configured so as to be written on with a pen.

14. A process according to claim 12, wherein the structured surface further comprises, a clock area by means of which a time can be marked on the structured surface.

15. A process according to claim 12, wherein the structured surface is a contiguous surface.

16. A process in accordance with claim 15, wherein the structured surface is a surface enclosing the first filter opening or the second filter opening in an at least partly circularly.

17. A process according to claim 12, wherein the structured surface comprises a product identification for the disposable filter.

18. A process according to claim 12, wherein a color of the imprint indicates a filter type of the disposable filter from a predefined group of filter types.

19. A process according to claim 12, wherein the structured surface comprises an iconographic symbol, which indicates an ability to write on the structured surface for a user of the disposable filter.

20. A process according to claim 12, wherein the disposable filter is selected from the group comprising an HME filter, an electrostatic filter, a mechanical filter and combinations thereof.
